(19) United States
(12) Patent Application Publication
(10) Pub. No.: US 2021/0403413 A1
(43) Pub. Date: Dec. 30, 2021

(54) PRODUCTION OF NITROGEN-CONTAINING CHELATORS

(71) Applicant: Ascend Performance Materials Operations LLC, Houston, TX (US)

(72) Inventors: Zhensheng Ding, Houston, TX (US); Huayun Yu, Houston, TX (US); Nick Sparkman, Houston, TX (US); Thomas Peavy, Houston, TX (US); Ernst Bezemer, Houston, TX (US); Kyle Kissell, Houston, TX (US)

(73) Assignee: Ascend Performance Materials Operations LLC, Houston, TX (US)

(21) Appl. No.: 17/363,808

(22) Filed: Jun. 30, 2021

Related U.S. Application Data
(60) Provisional application No. 63/046,180, filed on Jun. 30, 2020.

(51) Int. Cl. C07C 227/26; C07C 253/06

(52) U.S. Cl. CPC C07C 227/26; C07C 253/06

(57) ABSTRACT

Reaction pathways and conditions for the production of nitrogen-containing chelators, such as a glycine derivative, as described herein. In particular, the present disclosure describes a process for the production of a nitrile intermediate by reacting a tetra-amino compound with an aldehyde and a hydrogen cyanide to form the nitrile intermediate. The nitrile intermediate may then be further processed to produce the chelators at a high yield and/or a high purity.

### CROSS REFERENCE TO RELATED APPLICATIONS

This application claims priority to U.S. Provisional Application No. 63/046,180, filed Jun. 30, 2020, which is incorporated herein by reference.

### FIELD

The present disclosure relates generally to the production of nitrogen-containing chelators. In particular, the present disclosure relates to reaction pathways and conditions for the production of nitrogen-containing chelators with high yield and/or purity.

### BACKGROUND

Chelators, also known as chelating agents, are organic compounds whose structures allow them to form bonds to a metal atom. Because chelators typically form two or more separate coordinate bonds to a single, central metal atom, chelators can be described as polydentate ligands. Chelators often include sulfur, nitrogen, and/or oxygen, which act as electron-donating atoms in bonds with the metal atom.

Chelators are useful in a variety of applications, where their propensity to form chelate complexes with metal atoms is important. Conventional uses of chelators include in nutritional supplements, in medical treatments (e.g., chelation therapy to remove toxic metals from the body), as contrast agents (e.g., in MRI scans), in domestic and/or industrial cleaners and/or detergents, in the manufacture of catalysts, in removal of metals during water treatment, and in fertilizers. For example, chelators play an important role in treatment of cadmium or mercury poisoning, because the chelators can be selected to selectively bind to the metals and facilitate excretion.

Conventional chelators include, for example, aminopolyphosphonates, polycarboxylates, ethylenediaminetetraacetic acid (EDTA), diethylenetriaminepentaacetic acid (DTPA), nitrilotriacetic acid (NTA). These and other conventional chelators, however, exhibit a number of undesirable properties. Some conventional chelators do not demonstrate adequate activity or stability across a wide pH and/or temperature range. Some conventional chelators exhibit an unacceptably high toxicity. Some conventional chelators do not exhibit adequate solubility in aqueous and/or organic solvents. Some conventional chelators have low biodegradability and present high environmental risk.

Glycine derivatives, such as alanine-N,N-diacetonitrile, are a class of chelators that may exhibit these desirable properties. These chelators, which may be structural derivatives of the amino acid glycine, exhibit sufficient (or improved) activity and/or stability across a wide pH and/or temperature range, low toxicity, adequate solubility, and/or high biodegradability. Unfortunately, conventional processes, such as Strecker amino acid synthesis, for preparing glycine-derivative chelators are typically inefficient.

Thus, the need exists for processes for producing nitrile chelators and intermediates used to produce the nitrile chelators that demonstrate both efficiency and cost-effectiveness improvements. In particular, the need exists for producing glycine-derivative nitrile chelators without the need for a separate crystallization step. The resultant nitrile chelators and intermediates should have suitable (or improved) stability and activity across a wide pH and/or temperature range, low toxicity, and suitable biodegradability.

### SUMMARY

In one aspect, the present disclosure describes a process for preparing a nitrile intermediate, the process comprising: reacting a tetra-amino compound with a hydrogen cyanide and an aldehyde in an aqueous solution to form the nitrile intermediate. In some cases, the nitrile intermediate is formed at a yield greater than 5%. In some cases, the tetra-amino compound has a formula:

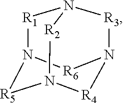

wherein R₁, R₂, R₃, R₄, R₅, and R₆ are independently (C₁-C₅)alkyl or (C₁-C₅)alkenyl. In some cases, R is (C₁-C₅)alkyl, and wherein R₁, R₂, R₃, R₄, R₅, and R₆ are independently (C₁-C₃)alkyl and more preferably CH₂. In some cases, the aldehyde is acetaldehyde. In some cases, the tetra-amino compound is 1,3,5,7-tetraazaadamantane. In some cases, the nitrile intermediate is alanine-N,N-dinitrile.

In some cases, the reacting comprises: providing a tetra-amino compound solution comprising the tetra-amino compound; adding the aldehyde to the tetra-amino compound solution to form a first intermediate solution; adjusting the pH of the first intermediate solution to a pH ranging from 2.0 to 7.0; adding the hydrogen cyanide to the first intermediate solution to form a second intermediate solution; heating and/or chilling the second intermediate solution to a first temperature; and heating and/or chilling the heated second intermediate solution to the second temperature. In some cases, the first temperature is from about 30° C. to about 80° C. In some cases, the second temperature is less than 25° C. In some cases, a temperature ramp rate from the first temperature to the second temperature is from 0.5° C./min to 5° C./min. In some cases, the hydrogen cyanide is added to the first intermediate solution at a rate from 0.01 g/minute to 1 g/minute. In some cases, the reacting comprises adding a nitrile intermediate seed to the reaction mixture. In some cases, the amount of nitrile intermediate seed added is less than 1% the theoretical yield of the nitrile intermediate.

In some cases, the process further comprises forming a glycine derivative from the nitrile intermediate. In some aspects, the glycine derivative has a formula

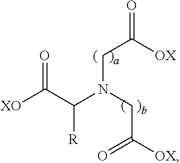

wherein: R is (C₁-C₁₀)alkyl, (C₁-C₁₀)haloalkyl, (C₁-C₁₀)alkenyl, or (C₁-C₁₀)alkyl carboxylate X is hydrogen, an alkali metal, an alkaline earth metal, or ammonium, a is from 0 to 5, and b is from 0 to 5. In some aspects, the forming the glycine derivative comprises hydrolyzing the nitrile intermediate. In some aspects, the hydrolyzing comprises reacting the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof. In some aspects, the glycine derivative is alanine-N,N-diacetic acid derivative. In some aspects, the glycine derivative is formed at a yield of at least 60%.

### DETAILED DESCRIPTION

### Introduction

As noted, the present disclosure describes reaction pathways and conditions for the production of nitrogen-containing chelator intermediates and the chelators, e.g., glycine derivatives, produced therefrom. In particular, the present disclosure describes a novel reaction scheme for the production of a nitrile intermediate. The nitrile intermediate may then be further processed to produce the chelators at a high yield and/or a high purity.

The present disclosure describes a novel process for preparing a nitrile intermediate from a tetra-amino compound. In particular, in the processes described herein, the nitrile intermediate is formed by reacting the tetra-amino compound with a hydrogen cyanide and an aldehyde (in an aqueous solution). The tetra-amino may have the structure:

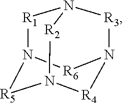

wherein R₁, R₂, R₃, R₄, R₅, and R₆ are independently (C₁-C₅)alkyl or (C₁-C₅)alkenyl. The aldehyde may have the formula R—CHO, where R is (C₁-C₁₀)alkyl, (C₁-C₁₀)haloalkyl, (C₁-C₁₀)alkenyl, or (C₁-C₁₀)alkyl carboxylate.

In some cases, the reaction pathways and conditions described herein advantageously produce the nitrile intermediate in crystalline form, e.g., without the need for a separate crystallization step. Conventional processes such as Strecker amino acid synthesis, in contrast, are inefficient and produce a nitrile intermediate in non-crystalline form (e.g., as an emulsion), which then requires an inefficient crystallization step. This is typically accomplished by complicated mechanical means, such as complex agitation procedures. The crystallization step reduces the efficiency of the overall reaction and provides further opportunity for the loss of product and/or the formation of impurities. The elimination of the need for crystallizing beneficially increases the efficiency of the reaction. For example, without the need for a separate crystallization step, the nitrile intermediate can be produced and collected more quickly. The crystalline nitrile intermediate also better facilitates conversion to the nitrogen-containing chelator. In addition, removing the crystallization step reduces the costs associated with the production of the nitrogen-containing chelators.

In some cases, a nitrile intermediate seed may be employed during the reaction, (e.g., the seed is added to one or more of the (intermediate) reaction mixtures of the reacting step). The nitrile intermediate seed has been found to beneficially promote the formation of the nitrile intermediate in crystalline form. As a result, the (crystalline) nitrile intermediate is surprisingly produced with high purity and/or high yield. Conventional processes do not employ nitrile intermediate seeds, and, as such, require significant additional processing to achieve crystallization (e.g., controlled agitation to produce crystals).

As discussed in detail below, the reacting the tetra-amino compound, hydrogen cyanide, and aldehyde to form the nitrile intermediate may take many forms. The reacting may comprise combining the reactants in an aqueous solution and allowing the reaction to proceed. In some cases, the reactants are combined substantially simultaneously. In some cases, the reactants are combined in particular order.

In some cases, the reacting comprises controlling the addition of the reactants as well as the reaction conditions. For example, in some embodiments, the various reactants may be added and/or combined in a specific order, and the nitrile intermediate seed may be added at specific points in the overall reaction scheme. Controlling the reaction according to the present disclosure may provide for increased yield and/or purity of the nitrile intermediate.

### Reactants

### Tetra-Amino Compound

According to the present disclosure, a nitrile intermediate is produced from a tetra-amino compound (as a reactant). The structure of the tetra-amino compound is not particularly limited, and any organic compound having at least four amino functional groups may be used. For example, the tetra-amino compound may comprise a saturated or unsaturated carbon chain having four or more amino functional groups. In some embodiments, the amino functional groups may be moieties of a carbon chain that comprises one or more heteroatoms, such as oxygen, sulfur, or phosphorus. The tetra-amino compound may be aliphatic or aromatic and may be open-chain (e.g., branched-chain, straight-chain) or cyclic (e.g., polycyclic).

In some embodiments, the tetra-amino compound is an aliphatic polycycle having four amino functional groups. For example, the tetra-amino compound may have a chemical structure:

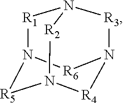

wherein R₁, R₂, R₃, R₄, R₅, and R₆ are independently (C₁-C₅)alkyl or (C₁-C₅)alkenyl. In some embodiments, the tetra-amino compound may have the above chemical structure, R₁, R₂, R₃, R₄, R₅, and R₆ are independently (C₁-C₃)alkyl. For example, R₁, R₂, R₃, R₄, R₅, and R₆ may be independently selected from a methylene group, an ethylene group, an n-propylene group, or an isopropylene group. In some embodiments, the tetra-amino compound may have the above chemical structure, wherein at least one of R₁, R₂, R₃, R₄, R₅, and R₆ is a methylene group, e.g., at least two, at least three, or at least four of R₁, R₂, R₃, R₄, R₅, and R₆ are methylene groups. Exemplary tetra-amino compounds according to the above chemical structure include tetraazaadamantane, methyl-tetraazaadamantane, dimethyl-tetraazaadamantane, trimethyl-tetraazaadamantane, tetramethyl-tetraazaadamantane, ethyl-tetraazaadamantane, diethyl-tetraazaadamantane, triethyl-tetraazaadamantane, tetraethyl-tetraazaadamantane, ethyl-methyl-tetraazaadamantane, propyl-tetraazaadamantane, dipropyl-tetraazaadamantane, tripropyl-tetraazaadamantane, tetrapropyl-tetraazaadamantane, and methyl-propyl-tetraazaadamantane.

In some embodiments, the tetra-compound is in a solid form. For example, the tetra-amino compound may be a powder.

In some embodiments, the tetra-amino compound may be dissolved in a solution, e.g., the tetra-amino compound may be a component of a tetra-amino compound solution (discussed in detail below). For example, the tetra-amino compound may be mixed with and/or dissolved in a solvent. For example, the process may include dissolving the tetra-amino compound in a solvent to prepare the tetra-amino compound solution. The composition of the tetra-amino compound solution is not particularly limited and may be any solution of the tetra-amino compound. In some embodiments, for example, the tetra-amino compound solution may comprise the tetra-amino compound dissolved in an aqueous solvent, e.g., water. In some embodiments, the tetra-amino compound solution may comprise the tetra-amino compound dissolved in an organic solvent. In some embodiments, the tetra-amino compound solution is a solution of the tetra-amino dissolved in a solvent system of both aqueous and organic solvents.

The concentration of the tetra-amino compound solution is not particularly limited. In some embodiments, the tetra-amino compound solution comprises from 1 wt. % to 65 wt. % of the tetra-amino compound, e.g., from 1 wt. % to 60 wt. %, from 1 wt. % to 55 wt. %, from 1 wt. % to 50 wt. %, from 1 wt. % to 45 wt. %, from 4 wt. % to 65 wt. %, from 4 wt. % to 60 wt. %, from 4 wt. % to 55 wt. %, from 4 wt. % to 50 wt. %, from 4 wt. % to 45 wt. %, from 8 wt. % to 65 wt. %, from 8 wt. % to 60 wt. %, from 8 wt. % to 55 wt. %, from 8 wt. % to 50 wt. %, from 8 wt. % to 45 wt. %, from 10 wt. % to 65 wt. %, from 10 wt. % to 60 wt. %, from 10 wt. % to 55 wt. %, from 10 wt. % to 50 wt. %, or from 10 wt. % to 45 wt. %. In terms of lower limits, the tetra-amino compound solution may comprise greater than 1 wt. % of the tetra-amino compound, e.g., greater than 4 wt. %, greater than 8 wt. %, or greater than 10 wt. %. In terms of upper limits, the tetra-amino compound solution may comprise less than 55 wt. % of the tetra-amino compound, e.g., less than 60 wt. %, less than 55 wt. %, less than 50 wt. %, or less than 45 wt. %.

### Aldehyde

The aldehyde may vary widely and many suitable aldehydes are known. In particular, the aldehyde may have a chemical formula R—CHO, where R is H, (C₁-C₁₀)alkyl, (C₁-C₁₀)haloalkyl, (C₁-C₁₀)alkenyl, or (C₁-C₁₀)alkyl carboxylate. In some embodiments, R of the aldehyde is (C₁-C₁₀)alkyl, e.g., (C₁-C₉)alkyl, (C₁-C₈)alkyl, (C₁-C₇)alkyl, (C₁-C₆)alkyl, or (C₁-C₅)alkyl. In some embodiments, R of the aldehyde is (C₁-C₁₀)haloalkyl, e.g., (C₁-C₉)haloalkyl, (C₁-C₈)haloalkyl, (C₁-C₇)haloalkyl, (C₁-C₆)haloalkyl, or (C₁-C₅)haloalkyl. a For example, the aldehyde may comprise a saturated or unsaturated, straight or branched carbon chain, e.g., a terminal carbonyl functional group. Exemplary aldehydes include acetaldehyde, propionaldehyde, butyraldehyde, pentanal, propenal, butenal, formyl ethanoic acid, formyl propionic acid, formyl butanoic acid, benzaldehyde, (4-methylphenyl)acetaldehyde, and (4-nitrophenyl)acetaldehyde.

As noted above, the order of the addition of the aldehyde to the other reactants may vary widely. In some cases, an aldehyde is added to and/or reacted with the tetra-amino compound (optionally in a heated tetra-amino compound solution) to form a first intermediate solution. For example, the aldehyde may be added to the tetra-amino compound solution, and/or the tetra-amino compound may be added to aldehyde. In some cases, the aldehyde may be added to a solution comprising the tetra-amino compound and the hydrogen cyanide.

In some embodiments, the nitrile intermediate seed is added to and/or reacted with the first intermediate solution to form a second intermediate solution. As noted, the nitrile intermediate seed may be added at other points in the reaction scheme, examples of which are discussed in more detail herein.

The amount of aldehyde used in the reaction, e.g., the amount aldehyde present in the first intermediate solution or the second intermediate solution, is not particularly limited. In some cases, excess aldehyde is used. In some cases, the amount of aldehyde used may be based on the amount of tetra-amino compound. In some embodiments, for example, an amount of aldehyde is added such that the molar ratio of the aldehyde to the tetra-amino compound is from 0.1:1 to 10:1, e.g., from 0.1:1 to 9:1, from 0.1:1 to 8:1, from 0.1:1 to 7:1, from 0.1:1 to 5:1, from 0.2:1 to 10:1, from 0.2:1 to 9:1, from 0.2:1 to 8:1, from 0.2:1 to 7:1, from 0.2:1 to 5:1, from 0.4:1 to 10:1, from 0.4:1 to 9:1, from 0.4:1 to 8:1, from 0.4:1 to 7:1, from 0.4:1 to 5:1, from 0.5:1 to 10:1, from 0.5:1 to 9:1, from 0.5:1 to 8:1, from 0.5:1 to 7:1, from 0.5:1 to 5:1, from 0.8:1 to 10:1, from 0.8:1 to 9:1, from 0.8:1 to 8:1, from 0.8:1 to 7:1, or from 0.8:1 to 5:1. In terms of lower limits, the molar ratio of the aldehyde to the tetra-amino compound may be greater than 0.1:1, e.g., greater than 0.2:1, greater than 0.4:1, greater than 0.5:1, or greater than 0.8:1. In terms of upper limits, the molar ratio of the aldehyde to the tetra-amino compound may be less than 10:1, e.g., less than 9:1, less than 8:1, less than 7:1, or less than 5:1.

### Hydrogen Cyanide

The hydrogen cyanide (HCN) is added to and/or reacted with one or more of the other reactants. The hydrogen cyanide may be combined with the tetra-amino compound before or after the aldehyde is introduced. In some embodiments, the aldehyde and the hydrogen cyanide are combined with the tetra-amino compound at substantially the same time, e.g., simultaneously or within several minutes of each other. In some embodiments, the hydrogen cyanide is added to and/or reacted with the tetra-amino compound solution.

The HCN, in some instances, may be employed in the form of a solution comprising HCN and the solution may be reacted with the tetra-amino compound (and the aldehyde) as described herein.

The amount of hydrogen cyanide used in the reaction, e.g., the amount hydrogen cyanide added to the tetra-amino compound solution, is not particularly limited. In some cases, excess hydrogen cyanide is used. In some cases, the amount of hydrogen cyanide used may be based on the amount of tetra-amino compound. In some embodiments, for example, an amount of hydrogen cyanide is added such that the molar ratio of the hydrogen cyanide to the tetra-amino compound is from 1:1 to 20:1, e.g., from 1:1 to 18:1, from 1:1 to 16:1, from 1:1 to 14:1, from 1:1 to 12:1, from 2:1 to 20:1, from 2:1 to 18:1, from 2:1 to 16:1, from 2:1 to 14:1, from 2:1 to 12:1, from 4:1 to 20:1, from 4:1 to 18:1, from 4:1 to 16:1, from 4:1 to 14:1, from 4:1 to 12:1, from 5:1 to 20:1, from 5:1 to 18:1, from 5:1 to 16:1, from 5:1 to 14:1, from 5:1 to 12:1, from 8:1 to 20:1, from 8:1 to 18:1, from 8:1 to 16:1, from 8:1 to 14:1, or from 8:1 to 12:1. In terms of lower limits, the molar ratio of the hydrogen cyanide to the tetra-amino compound may be greater than 1:1, e.g., greater than 2:1, greater than 4:1, greater than 5:1, or greater than 8:1. In terms of upper limits, the molar ratio of the hydrogen cyanide to the tetra-amino compound may be less than 20:1, e.g., less than 18:1, less than 16:1, less than 14:1, or less than 12:1.

### Nitrite Intermediate Seed

In some embodiments, the disclosed processes may employ a nitrile intermediate seed during the reaction. The timing of the addition of the nitrile intermediate seed to one or more of the reaction mixtures may vary. The addition of the nitrile intermediate seed has surprisingly been found to greatly improve the preparation of the nitrile intermediate, and subsequently the glycine derivative. In particular, the addition of the nitrile intermediate seed supports the formation of the nitrile intermediate (e.g., by the reaction of the dinitrile compound, the aldehyde, and the hydrogen cyanide) in crystalline form. Said another way, in some cases, the processes described herein produce crystalline nitrile intermediate due to the addition of the nitrile intermediate seed during the reaction. Furthermore, the formation of crystals in situ during the reaction contributes to improved yield and purity of the nitrile intermediate produced by the reaction.

Generally, the nitrile intermediate seed is an organic compound having at least two nitrile, or cyano, functional groups and at least one carboxyl functional group. Exemplary nitrile intermediates include alanine-N,N-diacetonitrile, alanine-N,N-dipropionitrile, alanine-N,N-dibutyronitrile, alanine-N-acetonitrile-N-propionitrile, alanine-N-acetonitrile-N-butyronitrile, ethyl glycine-N,N-diacetonitrile, ethyl glycine-N,N-dipropionitrile, ethyl glycine-N,N-dibutyronitrile, ethyl glycine-N-acetonitrile-N-propionitrile, ethyl glycine-N-acetonitrile-N-butyronitrile, propyl glycine-N,N-diacetonitrile, propyl glycine-N,N-dipropionitrile, propyl glycine-N,N-dibutyronitrile, propyl glycine-N-acetonitrile-N-propionitrile, and propyl glycine-N-acetonitrile-N-butyronitrile

In some embodiments, the chemical composition of the nitrile intermediate seed may be defined in relation to the nitrile intermediate to be formed by the reaction. For example, the nitrile intermediate seed may comprise substantially the same chemical structure (e.g., the same or slightly modified chemical structure) as the nitrile intermediate. Thus, any composition of the nitrile intermediate (discussed in detail below) may be used as the nitrile intermediate seed. The nitrile intermediate seed may be solid of the nitrile intermediate or may be a liquid solution comprising the nitrile intermediate. In some embodiments, for example, the nitrile intermediate seed is a solid crystal of the nitrile intermediate.

In the processes described herein, the nitrile intermediate seed is added to a reaction mixture before and/or during the reaction step. In some embodiments, the nitrile intermediate seed may combined with the dinitrile compound (e.g., the nitrile intermediate seed may be added to the dinitrile compound solution before the addition of both the aldehyde and the hydrogen cyanide). In some embodiments, the nitrile intermediate seed is added to the reaction mixture after the addition of the aldehyde (and before addition of the hydrogen cyanide). For example, the nitrile intermediate seed may be combined with the first intermediate solution (e.g., comprising the dinitrile compound and the aldehyde) to produce a second intermediate solution. In some embodiments, the nitrile intermediate seed is added to the reaction mixture after the addition of the hydrogen cyanide. In some embodiments, the nitrile intermediate seed is added to the reaction mixture at substantially the same time as the aldehyde and/or the hydrogen cyanide.

In some cases, only a small amount of the nitrile intermediate seed is required to produce the effects described herein, but larger amounts are contemplated. The amount of nitrile intermediate seed added to the reaction mixture may be described by reference to the theoretical yield of the nitrile intermediate by the reaction. In some embodiments, the amount of nitrile intermediate seed added to the reaction mixture is less than 1% of the theoretical yield of the nitrile intermediate by the reaction, e.g., less than 0.8%, less than 0.5%, less than 0.2%, less than 0.1%, or less than 0.08%. In terms of lower limits, the amount of nitrile intermediate added to the reaction mixture is greater than 0.0001% the theoretical yield of the nitrile intermediate by the reaction, e.g., greater than 0.0005%, greater than 0.001%, greater than 0.005%, or greater than 0.008%.

The amount of nitrile intermediate seed added to the reaction mixture may also be described by reference to the weight percentage of the nitrile intermediate seed in the reaction mixture (e.g., the weight percentage of the nitrile intermediate seed in the second intermediate solution). In some embodiments, the second intermediate solution comprises from 0.001 wt. % to 1 wt. % of the nitrile intermediate seed, e.g., from 0.001 wt. % to 0.5 wt. %, from 0.001 wt. % to 0.1 wt. %, from 0.001 wt. % to 0.1 wt. %, from 0.001 wt. % to 0.08 wt. %, from 0.005 wt. % to 1 wt. %, from 0.005 wt. % to 0.5 wt. %, from 0.005 wt. % to 0.1 wt. %, from 0.005 wt. % to 0.1 wt. %, from 0.005 wt. % to 0.08 wt. %, from 0.008 wt. % to 1 wt. %, from 0.008 wt. % to 0.5 wt. %, from 0.008 wt. % to 0.1 wt. %, from 0.008 wt. % to 0.1 wt. %, from 0.008 wt. % to 0.08 wt. %, from 0.01 wt. % to 1 wt. %, from 0.01 wt. % to 0.5 wt. %, from 0.01 wt. % to 0.1 wt. %, from 0.01 wt. % to 0.1 wt. %, or from 0.01 wt. % to 0.08 wt. %. In terms of upper limits, the second intermediate solution may comprise less than 1 wt. % nitrile intermediate seed, e.g., less than 0.5 wt. %, less than 0.1 wt. %, or less than 0.08 wt. %.

### Reaction

As noted above, the reacting step of the processes described herein may comprise controlling the addition of the reactants as well as the reaction conditions. For example, in some embodiments, the various reactants may be added and/or combined in a specific order, and the nitrile intermediate seed may be added at specific points in the overall reaction scheme. The reaction conditions described herein may further improve the production of the nitrile intermediate, e.g., the purity and/or yield of the nitrile intermediate. In particular, the present disclosure provides temperature and pH conditions, which the present inventors have surprisingly found produce the purity and/or the yield of the nitrile intermediate by the reaction described herein.

Controlling the reaction according to the present disclosure may provide for increased yield and/or purity of the nitrile intermediate.

In some cases, the tetra-amino compound solution may be provided for the reaction at any temperature or may be heated to a target temperature. In some embodiments, the tetra-amino compound solution is provided at room temperature. In some embodiments, the tetra-amino compound is from about 10° C. to about 30° C., e.g., from about 10° C. to about 29° C., from about 10° C. to about 28° C., from about 10° C. to about 27° C., from about 10° C. to about 26° C., from about 10° C. to about 25° C., from about 12° C. to about 30° C., from about 12° C. to about 29° C., from about 12° C. to about 28° C., from about 12° C. to about 27° C., from about 12° C. to about 26° C., from about 12° C. to about 25° C., from about 14° C. to about 30° C., from about 14° C. to about 29° C., from about 14° C. to about 28° C., from about 14° C. to about 27° C., from about 14° C. to about 26° C., from about 14° C. to about 25° C., from about 18° C. to about 30° C., from about 18° C. to about 29° C., from about 18° C. to about 28° C., from about 18° C. to about 27° C., from about 18° C. to about 26° C., from about 18° C. to about 25° C., from about 20° C. to about 30° C., from about 20° C. to about 29° C., from about 20° C. to about 28° C., from about 20° C. to about 27° C., from about 20° C. to about 26° C., from about 20° C. to about 25° C., from about 22° C. to about 30° C., from about 22° C. to about 29° C., from about 22° C. to about 28° C., from about 22° C. to about 27° C., from about 22° C. to about 26° C., or from about 22° C. to about 25° C.

In some cases, the reacting comprises adjusting the temperature of the aldehyde (or of the solution containing the aldehyde). In some embodiments, the aldehyde is heated or chilled (e.g., before combination with the tetra-amino compound solution) to a temperature from 1° C. to 40° C., e.g., from 2° C. to 35° C., from 3° C. to 30° C., or from 4° C. to 25° C. In some cases, the reacting comprises modifying (e.g., controlling and/or adjusting) the pH of the aldehyde (e.g., before combination with the tetra-amino compound solution) to a pH from 0.5 to 9.

As noted, the reacting may comprise adding the aldehyde (or a solution containing the aldehyde) to the tetra-amino compound solution, e.g., to form a first intermediate solution. The method of adding the aldehyde is not particularly limited. In some cases, for example, the aldehyde may be added to the tetra-amino compound solution by a syringe, e.g., a sub-surface syringe. In one embodiment, the aldehyde is added at a rate from 5 g/min to 25 g/min, e.g., from 8 g/min to 22 g/min, from 10 g/min to 20 g/min, or from 12 g/min to 15 g/min. In terms of lower limits, the addition rate may be greater than 5 g/min, e.g., greater than 8 g/min, greater than 10 g/min, or greater than 12 g/min. In terms of upper limits, the addition rate may be less than 25 g/min, e.g., less than 22 g/min, less than 20 g/min, less than 18 g/min, or less than 15 g/min.

In some cases, the reacting comprises adjusting the temperature of the hydrogen cyanide (or the solution containing the hydrogen cyanide). In some embodiments, the hydrogen cyanide is heated or chilled (e.g., before combination with the tetra-amino compound solution) to a temperature from 0° C. to 20° C., e.g., from 0.5° C. to 18° C., from 1° C. to 15° C., or from 1.5° C. to 10° C. In some cases, the reacting comprises modifying (e.g., controlling and/or adjusting) the pH of the hydrogen cyanide (e.g., before combination with the tetra-amino compound solution) to a pH from 0.5 to 9.

As noted, the reacting may comprise adding the hydrogen cyanide (or a solution containing the hydrogen cyanide) to the first intermediate solution, e.g., to form a second intermediate solution. The method of adding the hydrogen cyanide is not particularly limited. In some cases, for example, the hydrogen cyanide may be added to the tetra-amino compound solution by a syringe, e.g., a sub-surface syringe. In one embodiment, the hydrogen cyanide is added at a rate from 0.01 g/min to 1 g/min, e.g., from 0.02 g/min to 0.5 g/min, from 0.05 g/min to 0.3 g/min, or from 0.08 g/min to 0.2 g/min. In terms of lower limits, the addition rate may be greater than 0.01 g/min, e.g., greater than 0.02 g/min, greater than 0.05 g/min, or greater than 0.08 g/min. In terms of upper limits, the addition rate may be less than 1 g/min, e.g., less than 0.5 g/min, less than 0.3 g/min, or less than 0.2 g/min.

In some cases, reacting comprises combining the tetra-amino compound (e.g., the tetra-amino compound solution), the aldehyde, the hydrogen cyanide, and, optionally, the nitrile intermediate seed. In some embodiments, all reactants are combined simultaneously or substantially simultaneously (e.g., within a few minutes of each other). In some embodiments, the reactants are combined in a particular order. In some embodiments, for example, the tetra-amino compound (e.g., the tetra-amino compound solution) and the aldehyde may be combined before the addition of the hydrogen cyanide. In some embodiments, the tetra-amino compound (e.g., the tetra-amino compound solution) and the hydrogen cyanide may be combined before the addition of the aldehyde.

Mixtures, e.g., solutions, formed by the combining of two or more reactants, e.g., the first, second, or third intermediate solutions, may be referred to as reaction mixtures

The acidity and/or alkalinity of the reactants (and/or the reaction mixture and/or the various intermediate mixtures) can greatly affect the progress of the reaction. In particular, the reaction of the present disclosure may require an acidic environment (e.g., pH less than 7). In some cases, the reacting comprises modifying (e.g., controlling and/or adjusting) the pH of the a reaction mixture (e.g., the first intermediate solution and/or the second intermediate solution). In some embodiments, the pH can be modified by the addition of a mineral acid, e.g., hydrochloric acid, nitric acid, phosphoric acid, sulfuric acid, boric acid, hydrofluoric acid, hydrobromic acid, perchloric acid, or hydriodic acid.

In some embodiments, the pH of one or more reaction mixtures is controlled during the reaction. In some embodiments, the pH is maintained throughout the reaction. For example, the pH of the reaction may be adjusted (e.g., re-adjusted) after the reactants are combined, because the combination of the reactants may affect the pH of the reaction mixture. In some embodiments, the pH of the reaction mixture may be monitored (e.g., with a pH meter), and a mineral acid may be added to the reaction mixture if the measured pH increases with time.

In some embodiments, the reaction mixture is maintained at a pH ranging from 0 to 5, e.g., from 0 to 4.5, from 0 to 4 from 0 to 3.5, from 0 to 3, from 0.5 to 3, from 0.5 to 4.5, from 0.5 to 4 from 0.5 to 3.5, from 0.5 to 3, from 1 to 5, from 1 to 4.5, from 1 to 4 from 1 to 3.5, from 1 to 3, from 1.5 to 5, from 1.5 to 4.5, from 1.5 to 4 from 1.5 to 3.5, or from 1.5 to 3. In terms of lower limits, the pH of the reaction mixture may be maintained at greater than 0, e.g., greater than 0.5, greater than 1, or greater than 1.5 In terms of upper limits, the pH of the reaction mixture may be maintained at less than 5, e.g., less than 4.5, less than 4, less than 3.5, or less than 3.

The temperature of the reaction mixture can also greatly affect the process of the reaction. For example, the temperature of the reaction mixture may affect the solubility of the reactants in solution and/or the rate of the reaction. It is therefore desirable to control the temperature of the reaction mixture. As noted above, for example, each of the reactants (e.g., the tetra-amino compound solution, the acetaldehyde, and/or the hydrogen cyanide) may be heated or chilled before combining. In some embodiments, the temperature of the reaction mixture may also be controlled, or maintained. The method of controlling the temperature of the reaction mixture is not particularly limited. In some embodiments, a mechanical thermal control, e.g., a heat well or a hot plate, may be used to control the temperature of the reaction mixture.

In some embodiments, the temperature of one or more reaction mixtures is controlled during the reaction. In some embodiments, for example, the mixture of the tetra-amino compound (e.g., the tetra-amino compound solution), the aldehyde, the hydrogen cyanide is controlled. In some embodiments, the reaction mixture is heated or chilled during the addition of a reagent. For example, the reaction mixture may be heated during the addition of the aldehyde and/or the hydrogen cyanide.

In some embodiments, the reaction mixture is heated to a temperature from 35° C. and 100° C., e.g., from 35° C. to 95° C., from 35° C. to 90° C., from 35° C. to 85° C., from 35° C. to 80° C., from 40° C. and 100° C., from 40° C. to 95° C., from 40° C. to 90° C., from 40° C. to 85° C., from 40° C. to 80° C., from 45° C. and 100° C., from 45° C. to 95° C., from 45° C. to 90° C., from 45° C. to 85° C., from 45° C. to 80° C., from 50° C. and 100° C., from 50° C. to 95° C., from 50° C. to 90° C., from 50° C. to 85° C., from 50° C. to 80° C., from 55° C. and 100° C., from 55° C. to 95° C., from 55° C. to 90° C., from 55° C. to 85° C., from 55° C. to 80° C., from 60° C. and 100° C., from 60° C. to 95° C., from 60° C. to 90° C., from 60° C. to 85° C., or from 60° C. to 80° C. In terms of lower limits, the reaction mixture may be heated to a temperature greater than 35° C., e.g., greater than 40° C., greater than 45° C., greater than 50° C., greater than 55° C., or greater than 60° C. In terms of upper limits, the reaction mixture may be heated to a temperature less than 100° C., e.g., less than 95° C., less than 90° C., less than 85° C., or less than 80° C. In some cases, the reaction mixture may be heated to a temperature of about 60° C., about 81° C., about 82° C., about 83° C., about 84° C., about 85° C., about 86° C., about 87° C., about 88° C., about 89° C., about 70° C., about 71° C., about 72° C., about 73° C., about 74° C., about 76° C., about 77° C., about 78° C., about 79° C., about 80° C., about 81° C., about 82° C., about 83° C., about 84° C., or about 85° C., or a temperature therebetween.

The rate of heating the reaction mixture is not particularly limited. In one embodiment, for example, the reaction mixture is heated at a rate from 1° C./hour to 60° C./hour, e.g., from 1° C./hour to 50° C./hour, from 1° C./hour to 40° C./hour, from 1° C./hour to 30° C./hour, from 1° C./hour to 20° C./hour, from 3° C./hour to 60° C./hour, from 3° C./hour to 50° C./hour, from 3° C./hour to 40° C./hour, from 3° C./hour to 30° C./hour, from 3° C./hour to 20° C./hour, from 6° C./hour to 60° C./hour, from 6° C./hour to 50° C./hour, from 6° C./hour to 40° C./hour, from 6° C./hour to 30° C./hour, from 6° C./hour to 20° C./hour, from 10° C./hour to 60° C./hour, from 10° C./hour to 50° C./hour, from 10° C./hour to 40° C./hour, from 10° C./hour to 30° C./hour, from 10° C./hour to 20° C./hour, from 10° C./hour to 60° C./hour, from 10° C./hour to 50° C./hour, from 10° C./hour to 40° C./hour, from 10° C./hour to 30° C./hour, or from 10° C./hour to 20° C./hour.

The reaction mixture may be maintained at the heated temperature in order to ensure that the reaction runs to completion. In some cases, the reacting includes maintaining the heated temperature for a period of time. In some embodiments, for example, the heated temperature of the reaction mixture may be maintained for from 30 minutes to 180 minutes, e.g., from 30 minutes to 150 minutes, from 30 minutes to 120 minutes, from 30 minutes to 90 minutes, from 45 minutes to 180 minutes, from 45 minutes to 150 minutes, from 45 minutes to 120 minutes, or from 45 minutes to 90 minutes. In terms of lower limits, the heated temperature may be maintained for at least 30 minutes, at least 35 minutes, at least 40 minutes, at least 45 minutes, at least 50 minutes, or at least 55 minutes. In terms of upper limits, the heated temperature may be maintained for less than 180 minutes, e.g., less than 165 minutes, less than 150 minutes, less than 135 minutes, less than 120 minutes or less than 105 minutes. In some cases, the heated temperature is maintained for about 45 minutes, about 50 minutes, about 55 minutes, about 60 minutes, about 65 minutes, about 70 minutes, about 75 minutes, about 80 minutes, about 85 minutes, or about 90 minutes.

In some embodiments, the reacting comprises some combination of the above-described conditions and parameters. Said another way, the reacting may comprise any combination of the above described temperature, pH, and mixing parameters. In some embodiments, for example, the reacting may include providing a tetra-amino compound solution comprising the tetra-amino compound, adjusting the pH of the tetra-amino compound solution to a pH ranging from 0.5 to 4.0, heating the tetra-amino compound solution, adding the aldehyde to the heated tetra-amino compound solution to form a first intermediate solution, adding the nitrile intermediate seed to the first intermediate solution to form a second intermediate solution, adding hydrogen cyanide to the second intermediate solution to form a third intermediate solution, and/or heating the third intermediate solution to form the nitrile intermediate.

In some embodiments, the tetra-amino compound solution is adjusted to a pH ranging from 0.5 to 4, e.g., from 0.5 to 3.8, from 0.5 to 3.6, from 0.5 to 3.4, from 0.5 to 3.2, from 0.5 to 3.0, from 0.8 to 4, from 0.8 to 3.8, from 0.8 to 3.6, from 0.8 to 3.4, from 0.8 to 3.2, from 0.8 to 3.0, from 1.0 to 4, from 1.0 to 3.8, from 1.0 to 3.6, from 1.0 to 3.4, from 1.0 to 3.2, from 1.0 to 3.0, from 1.2 to 4, from 1.2 to 3.8, from 1.2 to 3.6, from 1.2 to 3.4, from 1.2 to 3.2, from 1.2 to 3.0, from 1.5 to 4, from 1.5 to 3.8, from 1.5 to 3.6, from 1.5 to 3.4, from 1.5 to 3.2, or from 1.5 to 3.0.

In some cases, reacting comprises combining the tetra-amino compound (e.g., the tetra-amino compound solution), the aldehyde, the hydrogen cyanide, and/or the nitrile intermediate seed. In some embodiments, all reactants are combined simultaneously or substantially simultaneously (e.g., within a few minutes of each other). In some embodiments, the reactants are combined in a particular order. In some embodiments, for example, the tetra-amino compound (e.g., the tetra-amino compound solution) and the aldehyde may be combined before the addition of the hydrogen cyanide. In some embodiments, the tetra-amino compound (e.g., the tetra-amino compound solution) and the hydrogen cyanide may be combined before the addition of the aldehyde.

Mixtures, e.g., solutions, formed by the combining of two or more reactants, e.g., the first, second, or third intermediate solutions, may be referred to as reaction mixtures

In some embodiments, the pH of one or more reaction mixtures is controlled during the reaction. As noted above, the reaction of the present disclosure may be conducted in an acidic environment. In some embodiments, the pH is maintained throughout the reaction. For example, the pH of the reaction may be adjusted (e.g., re-adjusted) after the reactants are combined, because the combination of the reactants may affect the pH of the reaction mixture. In some embodiments, the pH of the reaction mixture may be monitored (e.g., with a pH meter), and a mineral acid may be added to the reaction mixture if the measured pH increases with time.

In some embodiments, the reaction mixture is maintained at a pH ranging from 0.5 to 4, e.g., from 0.5 to 3.8, from 0.5 to 3.6, from 0.5 to 3.4, from 0.5 to 3.2, from 0.5 to 3.0, from 0.8 to 4, from 0.8 to 3.8, from 0.8 to 3.6, from 0.8 to 3.4, from 0.8 to 3.2, from 0.8 to 3.0, from 1.0 to 4, from 1.0 to 3.8, from 1.0 to 3.6, from 1.0 to 3.4, from 1.0 to 3.2, from 1.0 to 3.0, from 1.2 to 4, from 1.2 to 3.8, from 1.2 to 3.6, from 1.2 to 3.4, from 1.2 to 3.2, from 1.2 to 3.0, from 1.5 to 4, from 1.5 to 3.8, from 1.5 to 3.6, from 1.5 to 3.4, from 1.5 to 3.2, or from 1.5 to 3.0.

The temperature of the reaction mixture can also greatly affect the process of the reaction. For example, the temperature of the reaction mixture may affect the solubility of the reactants in solution and/or the rate of the reaction. It is therefore desirable to control the temperature of the reaction mixture. As noted above, for example, each of the reactants (e.g., the tetra-amino compound solution, the acetaldehyde, and/or the hydrogen cyanide) may be heated before combining. In some embodiments, the temperature of the reaction mixture may also be controlled, or maintained. The method of controlling the temperature of the reaction mixture is not particularly limited. In some embodiments, a mechanical thermal control, e.g., a heat well or a hot plate, may be used to control the temperature of the reaction mixture. Easymax from Mettler Toledo is an example of a suitable, commercially available mechanical thermal control.

In some embodiments, the temperature of one or more reaction mixtures is controlled during the reaction. In some embodiments, for example, the mixture of the tetra-amino compound (e.g., the tetra-amino compound solution), the aldehyde, the hydrogen cyanide, and the nitrile intermediate seed is heated. In some embodiments, the reaction mixture is heated to a temperature (e.g., a first temperature) from 30° C. and 80° C., e.g., from 30° C. to 75° C., from 30° C. to 70° C., from 30° C. to 65° C., from 30° C. to 60° C., from 32° C. and 80° C., from 32° C. to 75° C., from 32° C. to 70° C., from 32° C. to 65° C., from 32° C. to 60° C., from 35° C. and 80° C., from 35° C. to 75° C., from 35° C. to 70° C., from 35° C. to 65° C., from 35° C. to 60° C., from 38° C. and 80° C., from 38° C. to 75° C., from 38° C. to 70° C., from 38° C. to 65° C., from 38° C. to 60° C., from 40° C. and 80° C., from 40° C. to 75° C., from 40° C. to 70° C., from 40° C. to 65° C., from 40° C. to 60° C., from 42° C. and 80° C., from 42° C. to 75° C., from 42° C. to 70° C., from 42° C. to 65° C., or from 42° C. to 60° C. In terms of lower limits, the reaction mixture may be heated to a temperature greater than 30° C., e.g., greater than 32° C., greater than 35° C., greater than 38° C., greater than 40° C., or greater than 42° C. In terms of upper limits, the reaction mixture may be heated to a temperature less than 80° C., e.g., less than 75° C., less than 70° C., less than 65° C., or less than 60° C. In some cases, the reaction mixture may be heated to a temperature of about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 56° C., about 57° C., about 58° C., about 59° C., about 60° C., about 61° C., about 62° C., about 63° C., about 64° C., or about 65° C.

The rate of heating the reaction mixture is not particularly limited. In one embodiment, for example, the reaction mixture is heated at a rate from 1° C./hour to 45° C./hour, e.g., from 1° C./hour to 40° C./hour, from 1° C./hour to 35° C./hour, from 1° C./hour to 30° C./hour, from 1° C./hour to 25° C./hour, from 3° C./hour to 45° C./hour, from 3° C./hour to 40° C./hour, from 3° C./hour to 35° C./hour, from 3° C./hour to 30° C./hour, from 3° C./hour to 25° C./hour, from 6° C./hour to 45° C./hour, from 6° C./hour to 40° C./hour, from 6° C./hour to 35° C./hour, from 6° C./hour to 30° C./hour, from 6° C./hour to 25° C./hour, from 10° C./hour to 45° C./hour, from 10° C./hour to 40° C./hour, from 10° C./hour to 35° C./hour, from 10° C./hour to 30° C./hour, from 10° C./hour to 25° C./hour, from 10° C./hour to 45° C./hour, from 10° C./hour to 40° C./hour, from 10° C./hour to 35° C./hour, from 10° C./hour to 30° C./hour, or from 10° C./hour to 25° C./hour.

The reaction mixture may be maintained at the heated temperature in order to ensure that the reaction runs to completion. In some cases, the reacting includes maintaining the heated temperature for a period of time. In some embodiments, for example, the heated temperature of the reaction mixture may be maintained for from 30 minutes to 180 minutes, e.g., from 30 minutes to 150 minutes, from 30 minutes to 120 minutes, from 30 minutes to 90 minutes, from 45 minutes to 180 minutes, from 45 minutes to 150 minutes, from 45 minutes to 120 minutes, or from 45 minutes to 90 minutes. In terms of lower limits, the heated temperature may be maintained for at least 30 minutes, at least 35 minutes, at least 40 minutes, at least 45 minutes, at least 50 minutes, or at least 55 minutes. In terms of upper limits, the heated temperature may be maintained for less than 180 minutes, e.g., less than 165 minutes, less than 150 minutes, less than 135 minutes, less than 120 minutes or less than 105 minutes. In some cases, the heated temperature is maintained for about 45 minutes, about 50 minutes, about 55 minutes, about 60 minutes, about 65 minutes, about 70 minutes, about 75 minutes, about 80 minutes, about 85 minutes, or about 90 minutes.

In some embodiments, the process described herein includes modulating the temperature of the reaction mixture. For example, the reaction mixture may be heated and/or chilled to the first temperature and maintained at that temperature (as described above), and afterward the reaction mixture may be heated and/or chilled to a second temperature. In some cases, the temperature modulation ensures that the reaction is driven to completion. In some cases, the temperature modulation facilitated the formation of crystals.

In embodiments including temperature modulation, the second temperature is not particularly limited. In some embodiments, the second temperature is from 0° C. to 40° C., e.g., from 0° C. to 30° C., from 0° C. to 25° C., from 0° C. to 20° C., from 1° C. to 40° C., from 1° C. to 30° C., from 1° C. to 25° C., from 1° C. to 20° C., from 2° C. to 40° C., from 2° C. to 30° C., from 2° C. to 25° C., from 2° C. to 20° C., from 3° C. to 40° C., from 3° C. to 30° C., from 3° C. to 25° C., or from 3° C. to 20° C. In terms of lower limits, the second temperature may be greater than 0° C., e.g., greater than 1° C., greater than 2° C., or greater than 3° C. In terms of upper limits, the second temperature may be less than 40° C., e.g., less than 30° C., less than 25° C., or less than 20° C.

In some embodiments, the reacting comprises some combination of the above-described conditions and parameters. Said another way, the reacting may comprise any combination of the above described temperature, pH, and mixing parameters. In some embodiments, for example, the reacting may include providing a tetra-amino compound solution comprising the tetra-amino compound, adding the aldehyde to the tetra-amino compound solution to form a first intermediate solution, adjusting the pH of the first intermediate solution to between 2.0 and 7.0, adding hydrogen cyanide to the first intermediate solution to form a second intermediate solution, heating the second intermediate solution to the first temperature to form the nitrile intermediate, and/or cooling the second intermediate solution to the second temperature to form crystals of the nitrile intermediate.

### Reaction Intermediate

In some cases, the reaction of the tetra-amino compound, the aldehyde, and the hydrogen cyanide may produce a reaction intermediate. For example, a reaction intermediate may be produced by a reaction between the tetra-amino compound and the aldehyde. The reaction intermediate may further produce the aforementioned nitrile intermediate. For example, the reaction intermediate may react with the one or more reactants (e.g., the tetra-amino compound, the aldehyde, and the hydrogen cyanide) to produce the nitrile intermediate. In some cases, the reaction intermediate reacts with aldehyde and/or hydrogen cyanide by Strecker synthesis to produce the nitrile intermediate.

The reaction intermediate is not particularly limited and will vary with the reactants (e.g., the tetra-amino compound, the aldehyde, and the hydrogen cyanide). Generally, the reaction intermediate is a dinitrile compound, e.g., an organic compound having at least two nitrile, or cyano (—C≡N), functional groups. For example, the dinitrile compound may comprise a saturated or unsaturated carbon chain having two or more nitrile functional groups. In some embodiments, the nitrile functional groups may be moieties of a carbon chain that comprises one or more heteroatoms, such as oxygen, nitrogen, sulfur, or phosphorus. In some embodiments, the reaction intermediate is a compound having the chemical structure:

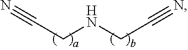

wherein a is from 0 to 5 and b is from 0 to 5. In some embodiments, the reaction intermediate is a dinitrile compound having the above chemical structure, wherein a is 1, and b is 0, 1, 2, 3, 4, or 5. In some embodiments, the dinitrile compound may have the above chemical structure, wherein a is 1 or 2, and be is 0, 1, 2, 3, or 4. In some embodiments, the dinitrile compound may have the above chemical structure, wherein a is 1, 2, or 3, and b is 1, 2, or 3. Exemplary dinitrile compounds according to the above chemical structure include ((cyanomethyl)amino)acetonitrile, ((cyanomethyl)amino)propanenitrile, ((cyanomethyl)amino)butanenitrile, ((cyanomethyl)amino)pentanenitrile, ((cyanoethyl)amino)acetonitrile, ((cyanoethyl)amino)propanenitrile, ((cyanoethyl)amino)butanenitrile, ((cyanoethyl)amino)pentanenitrile, ((cyanopropyl)amino)acetonitrile, ((cyanopropyl)amino)propanenitrile, ((cyanopropyl)amino)butanenitrile, ((cyanopropyl)amino)pentanenitrile, ((cyanobutyl)amino)acetonitrile, ((cyanobutyl)amino)propanenitrile, ((cyanobutyl)amino)butanenitrile, ((cyanobutyl)amino)pentanenitrile, ((cyanopropyl)amino)acetonitrile, ((cyanopropyl)amino)propanenitrile, ((cyanopropyl)amino)butanenitrile, and ((cyanopropyl)amino)pentanenitrile.

### Product, Nitrite Intermediate

As discussed above, the reaction of the tetra-amino compound, the aldehyde, and the hydrogen cyanide produces a nitrile intermediate. The nitrile intermediate is not particularly limited and will vary with the tetra-amino compound. Generally, the nitrile intermediate is an organic compound having at least two nitrile, or cyano, functional groups and at least one carboxyl functional group. In some embodiments, the nitrile intermediate is a compound having the chemical structure:

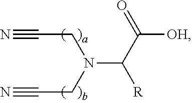

wherein a is from 0 to 5, b is from 0 to 5, and R is (C₁-C₁₀)alkyl, (C₁-C₁₀)haloalkyl, (C₁-C₁₀)alkenyl, or (C₁-C₁₀)alkyl carboxylate. In some embodiments, the nitrile intermediate may have the above chemical structure, wherein a is 1, and b is 0, 1, 2, 3, 4, or 5. In some embodiments, the nitrile intermediate may have the above chemical structure, wherein a is 1 or 2, and be is 0, 1, 2, 3, or 4. In some embodiments, the nitrile intermediate may have the above chemical structure, wherein a is 1, 2, or 3, and b is 1, 2, or 3. In some embodiments, R of the nitrile intermediate is (C₁-C₁₀)alkyl, e.g., (C₁-C₉)alkyl, (C₁-C₈)alkyl, (C₁-C₇)alkyl, (C₁-C₆)alkyl, or (C₁-C₅)alkyl. In some embodiments, R of the nitrile intermediate is (C₁-C₁₀)haloalkyl, e.g., (C₁-C₉)haloalkyl, (C₁-C₈)haloalkyl, (C₁-C₇)haloalkyl, (C₁-C₆)haloalkyl, or (C₁-C₅)haloalkyl. In some embodiments, R of the nitrile intermediate is (C₁-C₁₀)alkenyl, e.g., (C₂-C₁₀)alkenyl, (C₁-C₉)alkenyl, (C₂-C₉)alkenyl, (C₁-C₈)alkenyl, (C₂-C₈)alkenyl, (C₁-C₇)alkenyl, (C₂-C₇)alkenyl, (C₁-C₆)alkenyl, (C₂-C₆)alkenyl, (C₁-C₅)alkenyl or (C₂-C5)alkenyl. In some embodiments, R of the nitrile intermediate is (C₁-C₁₀)alkyl carboxylate, e.g., (C₁-C₉)alkyl carboxylate, (C₁-C₈)alkyl carboxylate, (C₁-C₇)alkyl carboxylate, (C₁-C₆)alkyl carboxylate, or (C₁-C₅)alkyl carboxylate. In particular, a and b may correspond to their respective values in the tetra-amino compound, and R may correspond to its respective value in the aldehyde.

Exemplary nitrile intermediates include alanine-N,N-diacetonitrile, alanine-N,N-dipropionitrile, alanine-N,N-dibutyronitrile, alanine-N-acetonitrile-N-propionitrile, alanine-N-acetonitrile-N-butyronitrile, ethyl glycine-N,N-diacetonitrile, ethyl glycine-N,N-dipropionitrile, ethyl glycine-N,N-dibutyronitrile, ethyl glycine-N-acetonitrile-N-propionitrile, ethyl glycine-N-acetonitrile-N-butyronitrile, propyl glycine-N,N-diacetonitrile, propyl glycine-N,N-dipropionitrile, propyl glycine-N,N-dibutyronitrile, propyl glycine-N-acetonitrile-N-propionitrile, and propyl glycine-N-acetonitrile-N-butyronitrile.

As has been discussed, the processes described herein produce the nitrile intermediate in crystalline form. Said another way, crystals of the nitrile intermediate are produced by the described processes, in particular without need for a separate crystallization step. Furthermore, the nitrile intermediate does not form an emulsion and therefore does not require additional mechanical processing (e.g., agitation) to separate. The formation of the nitrile intermediate in crystalline form increases the efficiency of the production process by removing the need for an additional step (and eliminating the time and cost associated therewith).

In some embodiments, the reaction product comprises the nitrile intermediate in an amount of less than 30 wt. %, e.g., less than 20 wt. %, less than 10 wt. %, or less than 5 wt. %.

The reaction product may further comprise unreacted reaction intermediate, e.g., as an impurity and/or side product. In some embodiments, for example, the reaction product comprises reaction intermediate in an amount from 5 wt. % to 50 wt. %, e.g., from 5 wt. % to 40 wt. %, from 5 wt. % to 30 wt. %, from 5 wt. % to 20 wt. %, from 8 wt. % to 50 wt. %, from 8 wt. % to 40 wt. %, from 8 wt. % to 30 wt. %, from 8 wt. % to 20 wt. %, from 10 wt. % to 50 wt. %, from 10 wt. % to 40 wt. %, from 10 wt. % to 30 wt. %, from 10 wt. % to 20 wt. %, from 10 wt. % to 50 wt. %, from 10 wt. % to 40 wt. %, from 10 wt. % to 30 wt. %, or from 10 wt. % to 20 wt. %. In terms of lower limits, the reaction product may comprise the reaction intermediate in an amount greater than 5 wt. %, e.g., greater than 8 wt. %, greater than 10 wt. %, or greater than 12 wt. %. In terms of upper limits, the reaction product may comprise the reaction intermediate in an amount less than 50 wt. %, e.g., less than 40 wt. %, less than 30 wt. %, or less than 20 wt. %.

### Further Reaction

As discussed above, the present disclosure also provides reaction pathways that include preparing the glycine derivative, e.g., alanine-N,N-diacetic acid, from the nitrile intermediate formed by the processes described herein, e.g., alanine-N,N-dinitrile. The structure of the glycine derivative is not particularly limited. As its name suggests, the glycine derivative may be a structural derivative of the amino acid glycine. In particular, the glycine derivative may be any organic compound having at least one carboxy functional group and at least one amino functional group, wherein the carboxy functional group and the amino functional group are separated by one carbon atom. In some embodiments, the carbon atom separating carboxy and amino functional groups may be modified with additional moieties. In some embodiments, the nitrogen of the amino functional group may be modified with additional moieties.

In some embodiments, the glycine derivative is an organic compound having two carboxy-containing functional groups as moieties on the nitrogen atom of the amino functional group. For example, the glycine derivative may have a chemical structure:

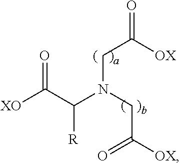

wherein a is from 0 to 5, b is from 0 to 5, and R is (C₁-C₁₀)alkyl, (C₁-C₁₀)haloalkyl, (C₁-C₁₀)alkenyl, or (C₁-C₁₀)alkyl carboxylate. In particular, a and b may correspond to their respective values in the dinitrile compound, and R may correspond to its respective value in the aldehyde. In the above chemical structure, X is hydrogen, an alkali metal, an alkaline earth metal, or ammonium. Exemplary nitrile intermediates include alanine-N,N-diacetic acid, alanine-N,N-dipropionic acid, alanine-N,N-dibutyric acid, alanine-N-acetic acid-N-propionic acid, alanine-N-acetic acid-N-butyric acid, ethyl glycine-N,N-diacetic acid, ethyl glycine-N,N-dipropionic acid, ethyl glycine-N,N-dibutyric acid, ethyl glycine-N-acetic acid-N-propionic acid, ethyl glycine-N-acetic acid-N-butyric acid, propyl glycine-N,N-diacetic acid, propyl glycine-N,N-dipropionic acid, propyl glycine-N,N-dibutyric acid, propyl glycine-N-acetic acid-N-propionic acid, and propyl glycine-N-acetic acid-N-butyric acid.

In the processes described herein, the glycine derivative may be formed by converting the nitrile functional groups of the nitrile intermediate to carboxy functional groups. In particular, the glycine derivative may be formed by hydrolyzing the nitrile intermediate.

Hydrolysis of the nitrile intermediate is not particularly limited and any known method may be used. In some embodiments, the hydrolysis is carried out in an aqueous solution using a strong acid. In some embodiments, the hydrolysis is carried out in an aqueous solution using a strong base. Suitable strong bases include inorganic bases, such as ammonium hydroxide, calcium hydroxide, lithium hydroxide, magnesium hydroxide, potassium hydroxide, sodium hydroxide, and combinations thereof.

The hydrolysis produces the glycine derivative at high yield. In some embodiments, the glycine derivative is formed at a yield greater than 60%, e.g., greater than 65%, greater than 70%, greater than 85%, greater than 90%. In terms of upper limits, the glycine derivative may be formed at a yield less than 100%, e.g., less than 99%, less than 98%, or less than 95%.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims or the equivalents thereof.

### EXAMPLES

The present disclosure will be further understood by reference to the following example.

Tetraazaadamantane (6.55 g) was added to 50 mL of deionized water at room temperature to produce a tetra-amino compound solution. Acetaldehyde (7.8 g) was added to the tetra-amino compound solution to form a first intermediate solution. Sulfuric acid was added to the dinitrile compound solution so as to adjust the pH of the first intermediate solution to 4.0. The acidified dinitrile compound solution was then heated to 30-35° C. Hydrogen cyanide (22 ml; about 15 g) was then added to the first intermediate solution over 90 minutes to form a second intermediate solution. While the hydrogen cyanide was being added, the second intermediate solution was heated to a first temperature 75° C. at a rate of about 0.3° C./minute.

After the addition of the hydrogen cyanide and temperature ramp was complete was complete, the second intermediate solution was cooled to 5° C. at a rate of about 2.3° C./minute. While the solution cooled, a crystalline reaction product formed. The solid crystals were filtered and dried overnight. A sample of the reaction product was analyzes and revealed that the reaction product comprised about 1.7 wt. % crystalline alanine-N,N-dinitrile (nitrile intermediate) and 14.6 wt. % ((cyanomethyl)amino)acetonitrile (reaction intermediate).

### Embodiments

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a process for preparing a nitrile intermediate, the process comprising: reacting a tetra-amino compound with a hydrogen cyanide and an aldehyde in an aqueous solution to form the nitrile intermediate.

Embodiment 2 is the process of embodiment(s) 1, wherein the nitrile intermediate is formed at a yield greater than 5%.

Embodiment 3 is the process according to any of the preceding embodiment(s), wherein the tetra-amino compound has a formula:

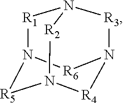

wherein R₁, R₂, R₃, R₄, R₅, and R₆ are independently (C₁-C₅)alkyl or (C₁-C₅)alkenyl.

Embodiment 4 is the process according to any of the preceding embodiment(s), wherein R is (C₁-C₅)alkyl, and wherein R₁, R₂, R₃, R₄, R₅, and R₆are independently (C₁-C₃)alkyl.

Embodiment 5 is the process according to any of the preceding embodiment(s), wherein the aldehyde is acetaldehyde.

Embodiment 6 is the process according to any of the preceding embodiment(s), wherein the tetra-amino compound is 1,3,5,7-tetraazaadamantane.

Embodiment 7 is the process according to any of the preceding embodiment(s), wherein the nitrile intermediate is alanine-N,N-dinitrile.

Embodiment 8 is the process according to any of the preceding embodiment(s), wherein the reacting comprises: providing a tetra-amino compound solution comprising the tetra-amino compound; adding the aldehyde to the tetra-amino compound solution to form a first intermediate solution; adjusting the pH of the first intermediate solution to a pH ranging from 2.0 to 7.0; adding the hydrogen cyanide to the first intermediate solution to form a second intermediate solution; heating and/or chilling the second intermediate solution to a first temperature; and heating and/or chilling the heated second intermediate solution to the second temperature.

Embodiment 9 is the process of embodiment(s) 8, wherein the first temperature is from about 30° C. to about 80° C.

Embodiment 10 is the process of embodiment(s) 8 or 9, wherein the second temperature is less than 25° C.

Embodiment 11 is the process according to any of embodiment(s) 8-10, wherein a temperature ramp rate from the first temperature to the second temperature is from 0.5° C./min to 5° C./min.

Embodiment 12 is the process according to any of embodiment(s) 8-11, wherein the hydrogen cyanide is added to the first intermediate solution at a rate from 0.01 g/minute to 1 g/minute.

Embodiment 13 is the process according to any of the preceding embodiment(s), wherein the reacting comprises adding a nitrile intermediate seed to the reaction mixture.

Embodiment 14 is the process according to embodiment 13, wherein the amount of nitrile intermediate seed added is less than 1% the theoretical yield of the nitrile intermediate.

Embodiment 15 is the process according to any of the preceding embodiment(s), further comprising forming a glycine derivative from the nitrile intermediate.

Embodiment 16 is the process of embodiment(s) 15, wherein the glycine derivative has a formula

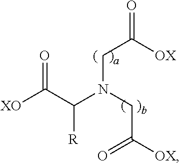

wherein: R is (C₁-C₁₀)alkyl, (C₁-C₁₀)haloalkyl, (C₁-C₁₀)alkenyl, or (C₁-C₁₀)alkyl carboxylate X is hydrogen, an alkali metal, an alkaline earth metal, or ammonium, a is from 0 to 5, and b is from 0 to 5.

Embodiment 17 is the process of embodiment(s) 15 or 16, wherein the forming the glycine derivative comprises hydrolyzing the nitrile intermediate.

Embodiment 18 is the process of embodiment(s) 17, wherein the hydrolyzing comprises reacting the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof.

Embodiment 19 is the process of any one of embodiment(s) 15-18, wherein the glycine derivative is alanine-N,N-diacetic acid derivative.

Embodiment 20 is the process of any one of embodiment(s) 15-18, wherein the glycine derivative is formed at a yield of at least 60%.

We claim:

1. A process for preparing a nitrile intermediate, the process comprising:
reacting a tetra-amino compound with a hydrogen cyanide and an aldehyde in an aqueous solution to form the nitrile intermediate.

2. The process of claim 1, wherein the nitrile intermediate is formed at a yield greater than 5%.

3. The process of claim 1, wherein the nitrile intermediate is crystalline.

4. The process of claim 1, wherein the tetra-amino compound has a formula: 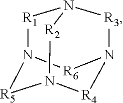
wherein R₁, R₂, R₃, R₄, R₅, and R₆ are independently (C₁-C₅)alkyl or (C₁-C₅)alkenyl.

5. The process of claim 1, wherein the aldehyde is acetaldehyde.

6. The process of claim 1, wherein the tetra-amino compound is 1,3,5,7-tetraazaadamantane.

7. The process of claim 1, wherein the nitrile intermediate is alanine-N,N-dinitrile.

8. The process of claim 1, wherein the reacting comprises:
providing a tetra-amino compound solution comprising the tetra-amino compound;
adding the aldehyde to the tetra-amino compound solution to form a first intermediate solution;
adjusting the pH of the first intermediate solution to a pH ranging from 2.0 to 7.0;
adding the hydrogen cyanide to the first intermediate solution to form a second intermediate solution;
heating and/or chilling the second intermediate solution to a first temperature; and
heating and/or chilling the heated second intermediate solution to the second temperature.

9. The process of claim 8, wherein the first temperature is from about 30° C. to about 80° C.

10. The process of claim 8, wherein the second temperature is less than 25° C.

11. The process of claim 8, wherein a temperature ramp rate from the first temperature to the second temperature is from 0.5° C./min to 5° C./min.

12. The process of claim 8, wherein the hydrogen cyanide is added to the first intermediate solution at a rate from 0.01 g/minute to 1 g/minute.

13. The process of claim 8, wherein the reacting comprises adding a nitrile intermediate seed to the reaction mixture.

14. The process of claim 13, wherein the amount of nitrile intermediate seed added is less than 1% the theoretical yield of the nitrile intermediate.

15. The process of claim 1, further comprising forming a glycine derivative from the nitrile intermediate.

16. The process of claim 15, wherein the glycine derivative has a formula 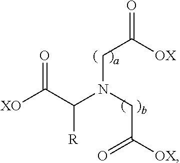
wherein:
R is (C₁-C₁₀)alkyl, (C₁-C₁₀)haloalkyl, (C₁-C₁₀)alkenyl, or (C₁-C₁₀)alkyl carboxylate
X is hydrogen, an alkali metal, an alkaline earth metal, or ammonium,
a is from 0 to 5, and
b is from 0 to 5.

17. The process of claim 15, wherein the forming the glycine derivative comprises hydrolyzing the nitrile intermediate.

18. The process of claim 17, wherein the hydrolyzing comprises reacting the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof.

19. The process of claim 15, wherein the glycine derivative is alanine-N,N-diacetic acid derivative.

20. The process of claim 15, wherein the glycine derivative is formed at a yield of at least 60%.
